(19) United States
(12) Patent Application Publication
(10) Pub. No.: US 2022/0192978 A1
(43) Pub. Date: Jun. 23, 2022

(54) Pharmaceutical Inhalation Aerosol and Preparation Method Therefor

(71) Applicant: CF PharmTech, Inc., Suzhou (CN)

(72) Inventors: MENG LI, Suzhou (CN); Hongfu TAO, Suzhou (CN); Yonghao MA, Suzhou (CN); Chenglin GAO, Suzhou (CN); Lili BOVET, Suzhou (CN)

(73) Assignee: CF PharmTech, Inc., Suzhou (CN)

(21) Appl. No.: 17/604,439

(22) PCT Filed: Jun. 4, 2020
(86) PCT No.: PCT/CN2020/094293

§ 371 Date: Oct. 18, 2021

(30) Foreign Application Priority Data
Jun. 26, 2019 (CN) 2019 10559969.5

(51) Int. Cl. A61K 9/00; A61K 9/12; A61K 9/16; A61K 31/40; A61K 31/4704

(52) U.S. Cl. CPC A61K 9/0073; A61K 9/12; A61K 31/4704; A61K 31/40; A61K 9/1682

(57) ABSTRACT

A pharmaceutical inhalation aerosol and a preparation method therefor. The preparation method comprises the following steps: (1) mixing glycopyrronium bromide coarse powder with indacaterol fine powder, or glycopyrronium bromide coarse powder with indacaterol coarse powder, or glycopyrronium bromide fine powder with indacaterol coarse powder in proportion to obtain a glycopyrronium bromide and indacaterol mixture; (2) micronizing, by a crushing device under pressure, the glycopyrronium bromide and indacaterol mixture prepared in step (1) to obtain a micronized glycopyrronium bromide and indacaterol mixture; and (3) adding the micronized glycopyrronium bromide and indacaterol mixture prepared in step (2) to an aluminum can, performing valve sealing, and filling with a propellant. In the glycopyrronium bromide/indacaterol compound inhalation aerosol prepared by the method, the effective deposition rate of glycopyrronium bromide is significantly improved, and the degree of co-deposition of glycopyrronium bromide and indacaterol is high. The prepared inhalation aerosol is convenient to carry and low in price, has higher medication compliance compared with an inhalation powder aerosol, and is more widely used than a nebulizer.

### FIELD OF THE INVENTION

The invention relates to a pharmaceutical inhalation aerosol and a preparation method therefor. In particular, the invention relates to a method for improving the Fine Particle Fraction (FPF) of glycopyrronium bromide, and increasing the degree of co-deposition of glycopyrronium bromide and indacaterol, in glycopyrronium bromide/indacaterol maleate inhalation aerosol.

### BACKGROUND OF THE INVENTION

Since the signing of the Montreal Convention, most aerosols using CFCs as propellants in the world have been withdrawn from the market. At present, the most commonly used inhalation aerosol propellants are HFA134a and HFA227. Inhalation aerosols have been used for many years to treat respiratory diseases, such as asthma, chronic obstructive pulmonary disease, and cystic fibrosis of lungs. In the development of inhalation aerosols, the physical and chemical properties (such as solubilities) of the Active Pharmaceutical Ingredients (APIs) determine whether the prepared inhalation aerosols are suspensions or solutions.

Inhalation aerosols use propellants as the power source for drug delivery. This is based on the fact that a propellant exists in liquid form in a container (mainly aluminum can). When the valve is pressed, the pharmaceutical formulation with the propellant as the vehicle is ejected from the nozzle, and the propellant quickly volatilizes to provide the kinetic energy for the forward horizontal movement of the drug particles. With the patient's inhalation, the drug particles can be delivered to the human bronchial and lung lesions to play a therapeutic role. The characteristics of inhalation aerosols are mainly as follows. Firstly, the production process is environmentally friendly and does not produce sewage with complex components. Secondly, solution-type inhalation aerosols mostly use ethanol as a co-solvent to increase the solubility of the API in the propellant, and sometimes can be added with glycerin (non-volatile co-solvent) and/or hydrochloric acid (pH adjuster). Suspension-type inhalation aerosols can be added with adjuvants including sodium cromoglycate (co-dispersant), oleic acid (surfactant ethanol (solubilizer), sorbitan trioleate (surfactant), PVP K25 (suspending agent), PEG1000 (valve lubricant). Thirdly, due to the high delivery efficiency of inhalation aerosols, 20-60% of the delivered dose directly enters the lung and bronchial lesions. Therefore, the dose of the drug in the tissues outside the lesions is small, so side effects are few, and the patient's drug compliance is high. Finally, inhalation aerosols are mainly used to treat various respiratory diseases, including but not limited to asthma, chronic obstructive pulmonary disease (COPD), and rhinitis. Inhalation aerosols are very suitable for this patient population, because these patients have poor respiratory function, and their inspiratory flow rate and flow are significantly lower than healthy people; while inhalation aerosols do not require rapid airflow to deliver the drug particles directly to the lung and bronchial lesions.

In addition, inhalation aerosols have the following advantages over dry powder inhalers. For capsule-type dry powder inhalers, the debris generated by piercing the capsule during administration may irritate the patient's throat. Reservoir-type dry powder inhalers are expensive, highly hygroscopic, have more demanding storage conditions (such as away from light), and have a short shelf life (for example, Seretide must be used up within 1 month after opening). In contrast, inhalation aerosols do not have the above problems, and patients have high drug compliance with them. Inhalation nebulizers need to be assembled and used with atomizers, atomizing cups, and liquid drugs. They cannot be carried around, are inconvenient to use, and are mainly used for children and the elderly, which have limitations in use, In contrast, Inhalation aerosols are suitable for patients of almost all age groups. Therefore, the development of compound inhalation aerosols with high Fine Particle Fraction has very important clinical value.

The active ingredients in inhalation aerosols currently on the market that can be used to treat respiratory diseases are mainly hormones and bronchodilators, and there are single, compound, and tripartite preparations. According to the dispersion system, inhalation aerosols can be divided into solution type, suspension type and emulsion type.

Single inhalation aerosols include fluticasone propionate aerosol (Flixotide®, GlaxoSmithKline), beclomethasone aerosol (QVAR®, Teva), beclomethasone aerosol (Clenil Modulite®, Chiesi), salmeterol aerosol (SEREVENT®, GlaxoSmithKline), formoterol aerosol (Atimos Modulite®, Chiesi), mometasone furoate aerosol (ASMANEX HFA®, Merck), salbutamol aerosol (Ventolin®, GlaxoSmithKline), ipratropium bromide aerosol (Atrovent HFA®, Boehringer-Ingelheim). Among them, beclomethasone aerosol and formoterol aerosol are solution type, and other products are suspension type.

Compound inhalation aerosols include salmeterol/ticasone aerosol (Seretide®, GlaxoSmithKline), mometasone furoate/formoterol aerosol (Durela®, Merck), budesonide/formoterol aerosol (Symbicort®, AstraZeneca), fluticasone propionate/formoterol aerosol (Flutiform®, Napp), beclomethasone/formoterol aerosol (Foster®, Chiesi), glycopyrronium bromide/formoterol aerosol (BEVESPI AEROSPHERE®, Pearl Therapeutics). Among them, Only Foster® developed by Chiesi is a solution-type product.

At present, there is only Tribow® developed by Chiesi on the market being tripartite preparation, which is a solution-type product.

Solution-type inhalation aerosols are faced with the problems in the development that, after the APIs are dissolved in the propellant, they are highly sensitive to acid, alkali, oxidation, high temperature, and moisture, and are prone to degradation, resulting in a greatly reduced drug content. EP1157689 discloses a formulation development technology of beclomethasone/formoterol aerosol. In the formulation, ethanol is used as a co-solvent to dissolve the two APIs in the propellant, and hydrochloric acid is added to adjust the pH of the solution. In order to improve the chemical stability, a specially coated can is used, and the pH is adjusted in the range of 2-5.

The most common challenge encountered in the development of suspension-type inhalation aerosols is the dispersion of drug particles. APIs that are sensitive to temperature and moisture will agglomerate, flocculate, and are difficult to disperse, resulting in poor aerodynamic particle size distribution and reduced fine particle dose in the lung, failing to achieve the expected efficacy. In the long-term stability study, because some active ingredients have sufficient moisture control in the initial stage, the aerodynamic characteristics of the drug particles are good. However, over time, the gasket between the valve and the aluminum can may have poor sealing performance, resulting in increased moisture content, which affects the content of active ingredients and aerodynamic characteristics thereof.

U.S. Pat. No. 8,143,239 discloses a new formulation of budesonide and formoterol inhalation aerosol, which contains 0.001% PVP K25 and 0.3% PEG1000. PVP K25 as a suspending agent can fully improve the physical stability of the formulation. After shaking, the active ingredients are evenly dispersed in the propellant. The experimental results show that, one minute after shaking, most of the drug particles are still in suspension, and the physical stability is good. In addition, experiments show that the Fine particle Fractions (FPFs) of the two active ingredients determined in the long-term stability study are both maintained in the range of 55-60%, and the physical stability is good. In order to solve this problem, Astrazene uses PVP K25 at an appropriate concentration (0.001%) as a suspending agent. After PVP is dissolved in the propellant, the viscosity of the propellant is enhanced and the sedimentation rate of the two active ingredients is reduced.

CN1150890 discloses a new MDI formulation in which an unconventional adjuvant sodium cromoglycate or nedocromil is added. Sodium cromoglycate or nedocromil is clinically a mast cell stabilizer, which inhibits inflammatory cells from releasing inflammatory mediators and treats asthma. However, in this formulation, the trace amount of sodium cromoglycate or nedocromil is not used for treatment but as a functional adjuvant to inhibit the adhesion and agglomeration of the active ingredients in the suspension and improve its dispersion. The active ingredients in this patent are fluticasone propionate and formoterol fumarate. The agglomeration of the two active ingredients is inhibited by adding sodium cromoglycate or nedocromil. The principle is that after sodium cromoglycate and formoterol are mixed uniformly and added with a propellant, sodium cromoglycate forms a stable association with formoterol in the form of a salt.

U.S. Pat. No. 8,808,713 filed by Pearl Therapeutics in 2010 discloses a new MDI formulation with a long-acting muscarinic antagonist (LAMA) such as glycopyrronium bromide, tiotropium bromide, and umeclidinium Bromide, and a long-acting β₂ adrenergic receptor agonist (LABA) such as indacaterol, formoterol, salmeterol, and olodaterol, as the main active ingredients, and phospholipids as the suspending particles. After the phospholipids are spray-dried, they become porous. The density of these porous phospholipids is much lower than that of the propellant, so the buoyancy of the porous phospholipids in the propellant is very high, and the active ingredient LABA or LAMA can be well adsorbed on the surface of the porous phospholipids suspended in the propellant. Even with shaking, centrifugating or temperature fluctuating, LABA or LAMA is still adsorbed on the surface of porous phospholipid particles without significant sedimentation or agglomeration.

These patents solve the problem of agglomeration of active ingredients in a suspension type inhalation aerosol by adding adjuvants, but these adjuvants may enter the human body and cause a series of uncomfortable reactions. In contrast, aerosols without adjuvants are economical, environmentally friendly, greatly reduce labor costs and raw material costs, and are suitable for industrial production.

In the development of suspension-type MDI compound products, it is not only necessary to consider the problem that the active ingredients are easy to settle due to higher density than the propellant, and poor dispersion will cause the low Fine Particle Fraction, but also to consider the difference in the sedimentation behavior of the two active ingredients in the propellant due to the difference in density, which ultimately leads to a low degree of co-deposition of the two active ingredients in the product. The low degree of co-deposition is embodied in in-vitro studies as a significant difference in the deposition rate of the drugs on each plate of the Anderson Cascade Impactor (ACI)(=the deposition amount of each plate/the total amount collected by the Anderson cascade impactor). Then the difference in the deposition rate of active ingredients in clinical lesions seriously affects the efficacy and safety of the formulation.

In September 2013, the European Medicines Agency (EMA) approved the glycopyrronium bromide/indacaterol Dry Powder Inhaler (DPI) developed by Novartis for the treatment of chronic obstructive pulmonary disease (COPD). This product (trade name: Ultibro Breezhaler) was approved for marketing in China on Dec. 28, 2017. Ultibro Breezhaler is a capsule-type DPI. As mentioned earlier, compared with an inhalation aerosol, the inherent defects of the capsule-type DPI are mainly as follows. The puncture of a capsule by a needle may cause capsule fragments to fall into drug powders, and the capsule fragments will enter the human respiratory tract during inhalation and cause dry cough and foreign body sensation; while the inhalation aerosol does not have this risk. The development cost of DPI products, especially the cost of device designs is high, so the price is higher than that of inhalation aerosol. DPI products need to be stored below 25° C. and need to be moisture-proof, because once the drug powders in the capsule absorb water, the product will not be effectively delivered to the human lungs; while most aerosols do not need to be moisture-proof, and storage conditions are not so strict.

In the present invention, the applicant therefore has developed glycopyrronium bromide/indacaterol inhalation aerosol products. Glycopyrronium bromide is an anticholinergic drug that can dilate bronchus, indacaterol is a β₂ receptor agonist, and the compound preparation containing the two can effectively treat COPD. In the development of the formulation, a single glycopyrronium bromide MDI and a single indacaterol MDI have been prepared in parallel. Study shows that, the aerodynamic behavior of micronized indacaterol in single or compound preparations is good, and the Fine Particle Fraction (FPF) (lung) is as high as 40-50%; while micronized glycopyrronium bromide has a low Fine Particle Fraction of 15-20%, whether it is a single preparation or a compound preparation, and most of the drugs are deposited in the throat. Obviously, glycopyrronium bromide is easy to agglomerate, has poor dispersion performance, and has a low Fine Particle Fraction.

As mentioned earlier, in the development of glycopyrronium bromide/indacaterol inhalation aerosol, the difference in the sedimentation behavior of the two active ingredients in the propellant has resulted in a low degree of co-deposition.

In order to solve the problem of low FPF, firstly, the applicant tried to add. ethanol and oleic acid (or sorbitan trioleate) to the formulation, where oleic acid (or sorbitan trioleate) was used as a surfactant to improve the dispersion problem of glycopyrronium bromide, and ethanol was used as a co-solvent to ensure that oleic acid (or sorbitan trioleate) could be completely dissolved in the propellant HFA-134a. The ACI results have shown that the Fine Particle Fractions of the two active ingredients were not improved, but rather decreased.

Secondly, sodium cromoglycate was added to the formulation, and it expected that sodium cromoglycate could improve the dispersibility of glycopyrronium bromide while inhibiting its agglomeration. It was found that the Fine Particle Fractions of indacaterol and glycopyrronium bromide were both improved, yet insignificantly, and glycopyrronium bromide still had serious agglomeration.

Finally, the commonly used suspending agent PVP1000 was added to the formulation, and it was expected that the suspending agent would increase the viscosity of the propellant HFA134a and delay the sedimentation rate of glycopyrronium bromide after shaking. The study found that the Fine Particle Fraction of glycopyrronium bromide has not been significantly improved, the agglomeration was serious, and it could not be effectively dispersed after shaking.

Obviously, in the prior art, adding a suspending agent or a surfactant or a co-dispersing agent to the formulation cannot effectively solve the problem of the low Fine Particle Fraction caused by the serious agglomeration of glycopyrronium bromide. The Fine Particle Fraction is very important for the treatment of patients with lung diseases. The low Fine Particle Fraction means that most of the drugs are deposited in the throat, and only a small amount of the drugs enter the lung lesions, and then the risk of adverse reactions caused by the drugs deposited in the oropharynx is higher. High Fine Particle Fraction indicates high product delivery efficiency and low risk of adverse reactions.

Andrew Theophilus of GSI and his colleagues published an article “Co-deposition of salmeterol and fluticasone propionate by combination inhaler” in International Journal of Pharmaceutics as early as 2006, in which the results of the suspension-type compound inhalation aerosol of salmeterol and fluticasone propionate in the Anderson cascade impactor test were compared with the results of salmeterol inhalation aerosol and fluticasone propionate inhalation aerosol. It was found that the difference in the deposition rate of the two active ingredients of the compound product at each plate was even smaller, and this was confirmed by Raman imaging studies. Clinical trials have shown that the efficacy (such as the efficacy index, peak expiratory flow (PEF)) of salmeterol/fluticasone propionate compound inhalation aerosol is better than the combined administration of fluticasone propionate aerosol and salmeterol aerosol. More importantly, it is the high degree of co-deposition of the two active ingredients in the compound formulation (that is, the small difference in the deposition rate of each plate) that realizes the synergistic effect of salmeterol and fluticasone propionate in the same lesion, and thus greatly improving the efficacy. Therefore, the degree of co-deposition of the two active ingredients in the compound inhalation aerosol is another important factor affecting the efficacy. At present, there is no public information on how to improve the co-deposition degree of glycopyrronium bromide and indacaterol in a suspension-type compound inhalation aerosol product of glycopyrronium bromide and indacaterol. Therefore, the improvement of the degree of co-deposition of glycopyrronium bromide and indacaterol is another technical problem we face.

### SUMMARY OF THE INVENTION

In the invention, fine powder refers to powder with D90≤5 μm, and coarse powder refers to powder with D90≥10 μm.

In the invention, D90 is described as the volume of particles smaller than a certain particle size (x) accounts for 90% of the total volume of the particles, D50 is described as the volume of particles smaller than a certain particle size (x) accounts for 50% of the total volume of the particles, and D10 is described as the volume of particles smaller than a certain particle size (x) accounts for 10% of the total volume of the particles.

The present invention solves the problem of low Fine Particle Fraction due to the particle agglomeration caused by the physical and chemical properties and surface characteristics of glycopyrronium bromide itself and the problem of low degree of co-deposition of glycopyrronium bromide and indacaterol by mixing glycopyrronium bromide and indacaterol first and then co-micronizing, thereby obtaining glycopyrronium bromide/indacaterol compound inhalation aerosol.

We are surprised to find that when glycopyrronium bromide fine powder is separately prepared as a suspension-type inhalation aerosol, the Fine Particle Fraction is low; when indacaterol fine powder is separately prepared as a suspension-type inhalation aerosol, the Fine Particle Fraction is high; and when glycopyrronium bromide fine powder and indacaterol fine powder are mixed and prepared as a suspension-type inhalation aerosol, the Fine Particle Fractions of glycopyrronium bromide and indacaterol are low.

However, when glycopyrronium bromide and indacaterol are first manually mixed, then co-micronized, and prepared as a suspension-type inhalation aerosol, the Fine Particle Fractions of glycopyrronium bromide and indacaterol are both significantly increased.

The aerodynamic particle size distribution of the glycopyrronium bromide/indacaterol compound inhalation aerosol prepared by directly mixing (5:1) glycopyrronium bromide fine powder (D90=3.43 μm) and indacaterol fine powder (D90=3.84 μm) was compared with those (FPF of 24% and 45%, respectively) of the single aerosol prepared from glycopyrronium bromide fine powder and the single aerosol prepared from indacaterol fine powder, respectively. The results show that the FPF (28.48%) of glycopyrronium bromide in the glycopyrronium bromide/indacaterol compound inhalation aerosol is increased by only 2.8%, while the FPF (38.50%) of indacaterol in the glycopyrronium bromide/indacaterol compound inhalation aerosol is reduced by nearly 7%.

The invention solves the problem of low glycopyrronium bromide deposition rate in the glycopyrronium bromide/indacaterol compound inhalation aerosol. A preparation method of pharmaceutical inhalation aerosol of the invention comprises the following steps: (1) mixing glycopyrronium bromide coarse powder with indacaterol fine powder, or glycopyrronium bromide coarse powder with indacaterol coarse powder, or glycopyrronium bromide fine powder with indacaterol coarse powder at a ratio to obtain a glycopyrronium bromide and indacaterol mixture;

(2) micronizing, by a crushing device under pressure, the glycopyrronium bromide and indacaterol mixture prepared in step (1) to obtain a micronized glycopyrronium bromide and indacaterol mixture; and

(3) adding the micronized glycopyrronium bromide and indacaterol mixture prepared in step (2) to an aluminum can, performing valve sealing, and filling with a propellant.

In step (1), the ratio is 5:1 to 1:5 by mass; preferably, the ratio is 1:1 to 1:5 by mass; more preferably, the ratio is 1:5 by mass. The pressure of the micronization is 4-10 bar. Preferably, the pressure of the micronization is 8-10 bar. More preferably, the pressure of the micronization is 10 bar. The propellant is any one of HFA 134a, HFA 227, and HFA 152, or a mixture of HFA 134a, HFA 227, and HFA 152; preferably, the propellant is HFA-134a. In step (2), the feed rate of the glycopyrronium bromide and indacaterol mixture is 0.5-1.0 g/min, preferably 0.5 g/min. The crushing device is a ball mill, a jet mill, a high-pressure homogenizer, or a spray dryer; preferably, the crushing device is a jet mill.

The aluminum can is preferably a coated aluminum can, more preferably an FCP-coated aluminum can.

In step (2), the particle size D90 distribution range of the micronized glycopyrronium bromide and indacaterol mixture is 2.86 μm to 4.18 μm, the D50 distribution range is 1.42 μm to 1.86 μm, and the D10 distribution range is 0.58 μm to 0.66 μm. Preferably, the particle size D90 distribution range of the micronized glycopyrronium bromide and indacaterol mixture is 2.86 μm to 3.58 μm, the D50 distribution range is 1.42 μm to 1.68 μm, and the D10 distribution range is 0.58 μm to 0.62 μm. More preferably, the particle size D90 distribution range of the micronized glycopyrronium bromide and indacaterol mixture is 2.86 μm to 3.40 μm, the D50 distribution range is 1.42 μm to 1.53 μm, and the D10 distribution range is 0.58 μm to 0.61 μm.

When an Anderson Cascade Impactor test is performed on the pharmaceutical inhalation aerosol prepared in step (3), the FPF of glycopyrronium bromide ranges from 35% to 60%, and the FPF of indacaterol ranges from 35% to 65%. Preferably, the FIT of glycopyrronium bromide ranges from 45% to 60%, and the FPF of indacaterol ranges from 40% to 60%. More preferably, the FPF of glycopyrronium bromide ranges from 50% to 60%, and the FPF of indacaterol ranges from 55% to 60%.

We are further surprised to find that, for a suspension-type inhalation aerosol prepared by manually mixing glycopyrronium bromide and indacaterol and micronizing at higher pressure, it is unexpectedly discovered that during the Anderson cascade impactor test, the deposition rate(=the amount deposited on a certain plate/the total amount collected by the Anderson cascade impactor) of glycopyrronium bromide deposited on each plate is very close to indacaterol. Which means that the degree of co-deposition of glycopyrronium bromide and indacaterol in various parts of the lung and bronchus is high after the patient is administered, and it is expected that the synergistic effect of the two will exert a better effect in the future.

The comparison of the deposition rate of each ACI plate of the suspension-type inhalation aerosol prepared by mixing glycopyrronium bromide fine powder and indacaterol fine powder is shown in Table 1 below. The comparison of the deposition rate of each ACI plate of the suspension-type inhalation aerosols prepared by mixing glycopyrronium bromide coarse powder and indacaterol coarse powder, or glycopyrronium bromide coarse powder and indacaterol fine powder, or glycopyrronium bromide fine powder and indacaterol coarse powder at a ratio of 5:1 is shown in Table 2 below.

TABLE 1
The comparison of the deposition rate of each
ACI plate of glycopyrronium bromide coarse
powder and indacaterol coarse powder

| | Prepared by mixing glycopyrronium bromide | | |
|---|---|---|---|
| | fine powder + indacaterol fine powder (5:1) | | |
| | Glycopyrronium | | |
| Plate | bromide | Indacaterol | Deposition rate ratio |
| Stage | 3.65 | 2.57 | 1.42 |
| plate 0 | | | |
| Stage | 5.08 | 3.81 | 1.33 |
| plate 1 | | | |
| Stage | 8.37 | 6.83 | 1.23 |
| plate 2 | | | |
| Stage | 15.44 | 14.51 | 1.06 |
| plate 3 | | | |
| Stage | 6.42 | 9.82 | 0.65 |
| plate 4 | | | |
| Stage | 1.48 | 4.64 | 0.32 |
| plate 5 | | | |
| Stage | 0.37 | 1.66 | 0.22 |
| plate 6 | | | |
| Stage | 0.27 | 0.67 | 0.40 |
| plate 7 | | | |

TABLE 2
The comparison of the deposition rate of each ACT plate of glycopyrronium bromide fine powder and indacaterol coarse powder

| | Glycopyrronium bromide | | | Glycopyrronium bromide | | | Glycopyrronium bromide | | |
|---|---|---|---|---|---|---|---|---|---|
| | coarse | | | coarse | | | fine | | |
| | powder + indacaterol | | | powder + indacaterol | | | powder + indacaterol | | |
| | coarse powder (5:1) | | | fine powder (5:1) | | | coarse powder (5:1) | | |
| | Glycopyrronium | | Deposition | Glycopyrronium | | Deposition | Glycopyrronium | | Deposition |
| Plate | bromide | Indacaterol | rate ratio | bromide | Indacaterol | rate ratio | bromide | Indacaterol | rate ratio |
| Stage | 1.97 | 1.79 | 1.10 | 2.53 | 2.39 | 1.06 | 2.10 | 2.02 | 1.04 |
| plate | | | | | | | | | |
| 0 | | | | | | | | | |
| Stage | 2.30 | 2.11 | 1.09 | 2.74 | 2.50 | 1.10 | 2.07 | 1.98 | 1.05 |
| plate | | | | | | | | | |
| 1 | | | | | | | | | |
| Stage | 4.14 | 3.88 | 1.07 | 4.63 | 4.16 | 1.11 | 4.05 | 4.12 | 0.98 |
| plate | | | | | | | | | |
| 2 | | | | | | | | | |
| Stage | 15.55 | 15.27 | 1.02 | 16.83 | 16.09 | 1.05 | 17.19 | 17.51 | 0.98 |
| plate | | | | | | | | | |
| 3 | | | | | | | | | |
| Stage | 17.76 | 17.97 | 0.99 | 15.17 | 15.49 | 0.98 | 19.60 | 17.19 | 1.14 |
| plate | | | | | | | | | |
| 4 | | | | | | | | | |
| Stage | 7.30 | 8.15 | 0.90 | 5.49 | 7.15 | 0.77 | 7.10 | 6.96 | 1.02 |
| plate | | | | | | | | | |
| 5 | | | | | | | | | |
| Stage | 0.92 | 1.30 | 0.71 | 0.92 | 1.54 | 0.60 | 0.79 | 1.17 | 0.68 |
| plate | | | | | | | | | |
| 6 | | | | | | | | | |
| Stage | 0.27 | 0.42 | 0.64 | 0.33 | 0.49 | 0.68 | 0.28 | 0.40 | 0.7 |
| plate | | | | | | | | | |

The present invention relates to the research on the ratio of glycopyrronium bromide/indacaterol compound aerosol. The dispersibility of glycopyrronium bromide is poor, and the co-micronization process can can improve the dispersibility of glycopyrronium bromide under the action of indacaterol. Therefore, changes in the ratio of glycopyrronium bromide/indacaterol may affect the dispersibility of glycopyrronium bromide, which in turn affects the FPF of glycopyrronium bromide. The method is as described above.

Studies have shown that as the proportion of glycopyrronium bromide in glycopyrronium bromide powder and indacaterol powder decreases, the dispersibility of glycopyrronium bromide is significantly improved and FPF is increased. After different ratios of glycopyrronium bromide powder/indacaterol powder are co-micronized by crushing device under pressure, the degree of co-deposition of glycopyrronium bromide and indacaterol in the prepared aerosol is improved.

The invention also relates to the influence of pressure when glycopyrronium bromide/indacaterol is micronized by a jet mill. The method is as described above.

### Advantageous Effects

The invention adopts a brand-new solution to solve the problem of low Fine Particle Fraction of glycopyrronium bromide, and the problem of low co-deposition degree of glycopyrronium bromide and indacaterol, in glycopyrronium bromide/indacaterol compound inhalation aerosol.

The method of the present invention is only a physical process, which is simple and fast.

The glycopyrronium bromide/indacaterol compound inhalation aerosol prepared by the method of the present invention only contains the active ingredients glycopyrronium bromide, indacaterol, and a propellant, and co-solvents, suspending agents, surfactants and other adjuvants are not needed, thus the formulation is highly economical, green and environmentally friendly, greatly reduces labor costs and raw material costs, and is suitable for industrial production.

### BRIEF DESCRIPTION OF THE DRAWINGS

FIG. 1A shows the Anderson cascade impactor test results of an aerosol prepared by pulverizing glycopyrronium bromide coarse powder and indacaterol maleate coarse powder at a ratio of 5:1 with a jet mill;

FIG. 1B shows the Anderson cascade impactor test results of an aerosol prepared by pulverizing glycopyrronium bromide coarse powder and indacaterol maleate coarse powder at a ratio of 1:1 with a jet mill;

FIG. 1C shows the Anderson cascade impactor test results of an aerosol prepared by pulverizing glycopyrronium bromide coarse powder and indacaterol maleate coarse powder at a ratio of 1:5 with a jet mill;

FIG. 2A shows the Anderson cascade impactor test results of an aerosol prepared by pulverizing glycopyrronium bromide coarse powder and indacaterol maleate fine powder at a ratio of 5:1 with a jet mill;

FIG. 2B shows the Anderson cascade impactor test results of an aerosol prepared by pulverizing glycopyrronium bromide coarse powder and indacaterol maleate fine powder at a ratio of 1:1 with a jet mill;

FIG. 2C shows the Anderson cascade impactor test results of an aerosol prepared by pulverizing glycopyrronium bromide coarse powder and indacaterol maleate fine powder at a ratio of 1:5 with a jet mill;

FIG. 3A shows the Anderson cascade impactor test results of an aerosol prepared by pulverizing glycopyrronium bromide fine powder and indacaterol maleate coarse powder at a ratio of 5:1 with a jet mill;

FIG. 3B shows the Anderson cascade impactor test results of an aerosol prepared by pulverizing glycopyrronium bromide fine powder and indacaterol maleate coarse powder at a ratio of 1:1 with a jet mill;

FIG. 3C shows the Anderson cascade impactor test results of an aerosol prepared by pulverizing glycopyrronium bromide fine powder and indacaterol maleate coarse powder at a ratio of 1:5 with a jet mill;

FIG. 4A shows the Anderson cascade impactor test results of an aerosol prepared by pulverizing glycopyrronium bromide coarse powder and indacaterol maleate coarse powder at a ratio of 1:1 with a jet mill at a pressure of 10 bar;

FIG. 4B shows the. Anderson cascade impactor test results of an aerosol prepared by pulverizing glycopyrronium bromide coarse powder and indacaterol maleate coarse powder at a ratio of 1:1 with a jet mill at a pressure of 8 bar;

FIG. 4C shows the Anderson cascade impactor test results of an aerosol prepared by pulverizing glycopyrronium bromide coarse powder and indacaterol maleate coarse powder at a ratio of 1:1 with a jet mill at a pressure of 4 bar;

FIG. 4D shows the Anderson cascade impactor test results of an aerosol prepared by pulverizing glycopyrronium bromide coarse powder and indacaterol maleate coarse powder at a ratio of 1:1 with a jet mill at a pressure of 3 bar;

FIG. 4E shows the Anderson cascade impactor test results of an aerosol prepared by pulverizing glycopyrronium bromide coarse powder and indacaterol maleate coarse powder at a ratio of 1:1 with a jet mill at a pressure of 2 bar;

FIG. 5 shows the Anderson cascade impactor test results of a compound aerosol prepared from micronized glycopyrronium bromide powder and micronized indacaterol maleate powder.

### DETAILED DESCRIPTION OF THE INVENTION

In conjunction with the drawings and the preferred embodiments of the present invention, the following further describes the technical means adopted by the present invention to achieve the intended purpose of the invention.

Experimental equipment: jet mill: Micron JETMILL Lab ultra-fine powder jet mill; High performance liquid chromatography (HPLC) instrument: Waters 2695.

Reagent Source: glycopyrronium bromide was purchased from Harman Finochem Ltd, India; indacaterol maleate was purchased from Inke, Italy; and HFA 134a was purchased from (Japan) Mexichem.

Prescription amounts of glycopyrronium bromide and indacaterol maleate were added to an aluminum can, valve-sealed, and filled with propellant, HFA-134a. After being inverted for 2 days, it was tested according to 0951, volume IV general rules of the Pharmacopoeia of the People's Republic of China 2015 Edition. The sum of the deposition from the stage plate 3 to the filter membrane is the Fine Particle Dose (FPD). The FPD (less than 5 μm) divided by the total amount collected by the Anderson cascade impactor (Total Dose, TD) is the Fine Particle Fraction (FPF).

Experimental Method

Micronized powders were precisely weighed and placed in a 14 mL fluorocarbon polymerization (FCP)-coated aluminum can, valve-sealed, filled, ultrasonicated for 10 minutes, and then kept for 2 days for Anderson cascade impactor testing. It was tested according to 0951 device 2, volume IV general rules of the Pharmacopoeia of the People's Republic of China 2015 Edition. The relative humidity of the test environment should be 45% to 55%. The flow rate was adjusted to 28.3±1.5 liters per minute. Take 1 bottle of this product, shake it sufficiently, and discard 4 sprays. Wipe the mouthpiece with ethanol, dry it sufficiently, turn on the vacuum pump, shake for 5 seconds (note that shake for 5 seconds before each spray and the interval between sprays is 30 seconds), insert the product into the adapter, and spray once immediately. After removing the aluminum can and the driver, shake for 5 seconds, reinsert into the adapter, and immediately spray the second time. Repeat this process until the completion of 10 sprays. After the last spray, wait 1 minute, then remove the aluminum can and driver, turn off the vacuum pump, and remove the device. The various plates of the Anderson impactor were washed with a specific ratio of methanol aqueous solution, and the content of the drugs deposited on each plate was determined by HPLC.

### EXAMPLE 1

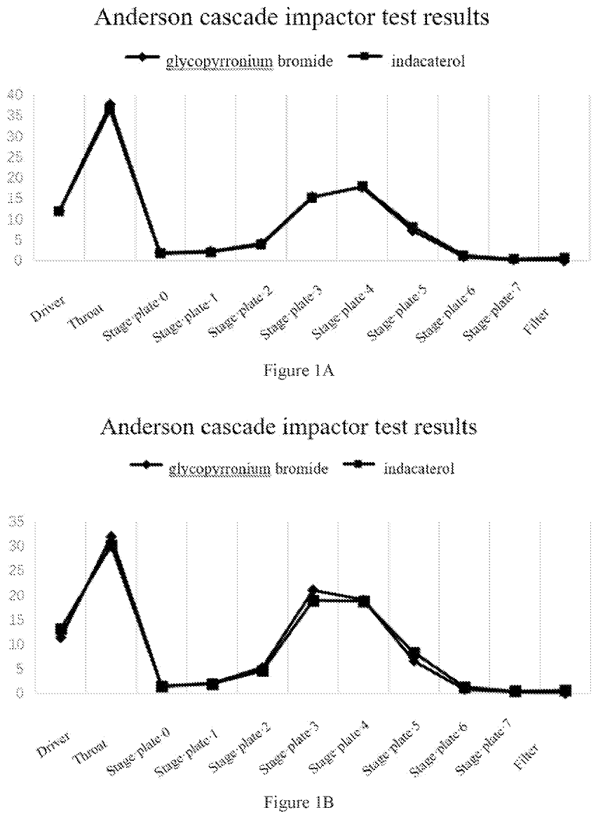

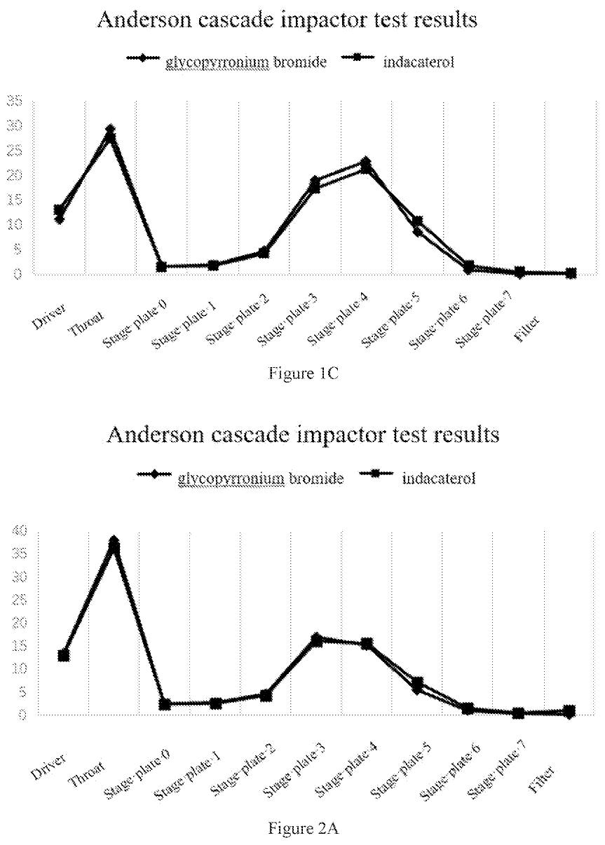

1.0 g glycopyrronium bromide coarse powder and 0.2 g indacaterol maleate coarse powder (5:1) were weighed and mixed manually for 10 minutes, then the mixture was added to a jet mill, and micronized at a pressure of 8 bar. The micronized API was sealed and stored until use. 24 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The ACI test method is as described above. The Anderson cascade impactor test results are shown in FIG. 1A.

0.5 g glycopyrronium bromide coarse powder and 0.5 g indacaterol maleate coarse powder (1:1) were weighed and mixed manually for 10 minutes, then the mixture was added to a jet mill, and micronized at a pressure of 8 bar. The micronized API was sealed and stored until use. 24 mg of the glycopyrronium bromide/indacaterol maleate mixture was weighed, placed in a 14 mL FCP-coated aluminum can, valve-sealed, filled, ultrasonicated for 10 minutes, and then kept for 2 days for testing. The Anderson cascade impactor test results are shown in FIG. 1B.

0.2 g glycopyrronium bromide coarse powder and 1.0 g indacaterol maleate coarse powder (1:5) were weighed and mixed manually for 10 minutes, then the mixture was added to a jet mill, and micronized at a pressure of 8 bar. The micronized API was sealed and stored until use. 30 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 1C.

TABLE 3
The Anderson cascade impactor test results of glycopyrronium
bromide coarse powder and indacaterol maleate coarse powder
after being co-micronized by a jet mill at a pressure of 8 bar

| | FPF of | FPF of |
|---|---|---|
| | glycopyrronium | indacaterol |
| Formulation | bromide (%) | maleate (%) |
| glycopyrronium bromide + | 47.55 | 49.80 |
| indacaterol maleate (5:1) | | |
| glycopyrronium bromide + | 54.03 | 56.11 |
| indacaterol maleate (1:1) | | |
| glycopyrronium bromide + | 58.03 | 59.71 |
| indacaterol maleate (1:5) | | |

It can be seen from Table 3 that at a pressure of 8 bar, the FPFs of glycopyrronium bromide and indacaterol maleate are 47.55% and 49.8% respectively when the ratio of glycopyrronium bromide/indacaterol maleate reaches 5:1; the FPFs of glycopyrronium bromide and indacaterol maleate are 54.03% and 56.11% respectively when the ratio increases to 1:1; and the FPFs of glycopyrronium bromide and indacaterol maleate are 58.03% and 59.71% respectively when the ratio increases to 1:5. As the proportion of glycopyrronium bromide decreases, the dispersibility of glycopyrronium bromide is significantly improved, and the Fine Particle Fraction is increased.

The Anderson test results in FIGS. 1A-1C show that for the aerosols prepared by co-micronizing glycopyrronium bromide coarse powder/indacaterol maleate coarse powder at different ratios with a jet mill at a pressure of 8 bar, the degree of co-deposition of glycopyrronium bromide and indacaterol maleate is very high.

### EXAMPLE 2

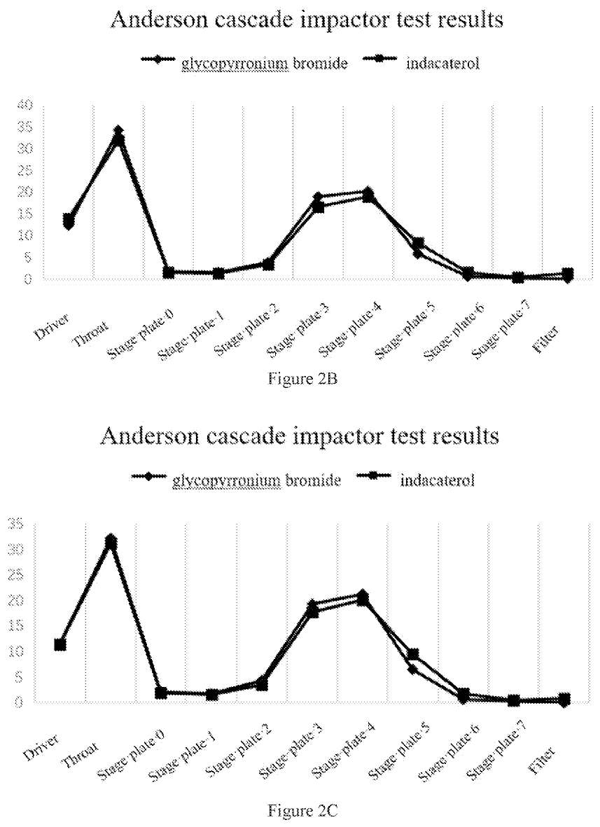

1.5 g glycopyrronium bromide coarse powder and 0.3 g indacaterol maleate fine powder (5:1) were weighed and mixed manually for 10 minutes, then the mixture was added to a jet mill, and micronized at a pressure of 8 bar. The micronized API was sealed and stored until use. 24 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 2A.

0.5 g glycopyrronium bromide coarse powder and 0.5 g indacaterol maleate fine powder (1:1) were weighed and mixed manually for 10 minutes, then the mixture was added to a jet mill, and micronized at a pressure of 8 bar. The micronized API was sealed and stored until use. 24 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 2B.

0.2 g glycopyrronium bromide coarse powder and 1.0 g indacaterol maleate fine powder (1:5) were weighed and mixed manually for 10 minutes, then the mixture was added to a jet mill, and micronized at a pressure of 8 bar. The micronized API was sealed and stored until use. 30 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 2C.

TABLE 4
The Anderson cascade impactor test results of glycopyrronium
bromide coarse powder and indacaterol maleate fine powder after
being co-micronized by a jet mill at a pressure of 8 bar

| | FPF of | |
|---|---|---|
| | glycopyrronium | FPF of indacaterol |
| Formulation | bromide (%) | maleate (%) |
| glycopyrronium bromide + | 44.88 | 47.98 |
| indacaterol maleate (5:1) | | |
| glycopyrronium bromide + | 52.77 | 55.38 |
| indacaterol maleate (1:1) | | |
| glycopyrronium bromide + | 54.45 | 56.90 |
| indacaterol maleate (1:5) | | |

As shown in Table 4, as the proportion of glycopyrronium bromide decreases, the dispersibility of glycopyrronium bromide is significantly improved, and the Fine Particle Fraction is increased from 44.88% to 54.45%.

The Anderson test results in FIGS. 2A-2C show that for the aerosols prepared by co-micronizing glycopyrronium bromide coarse powder/indacaterol maleate fine powder at different ratios with a jet mill at a pressure of 8 bar, the degree of co-deposition of glycopyrronium bromide and indacaterol maleate is very high.

### EXAMPLE 3

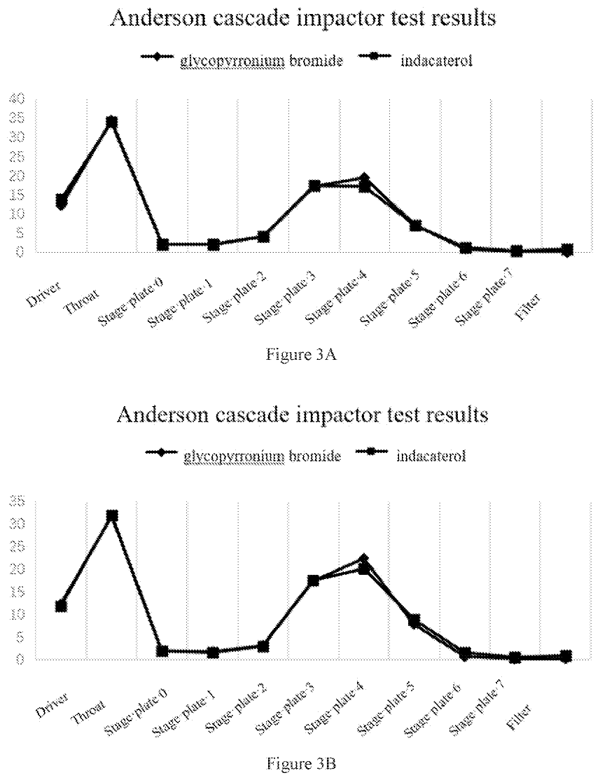

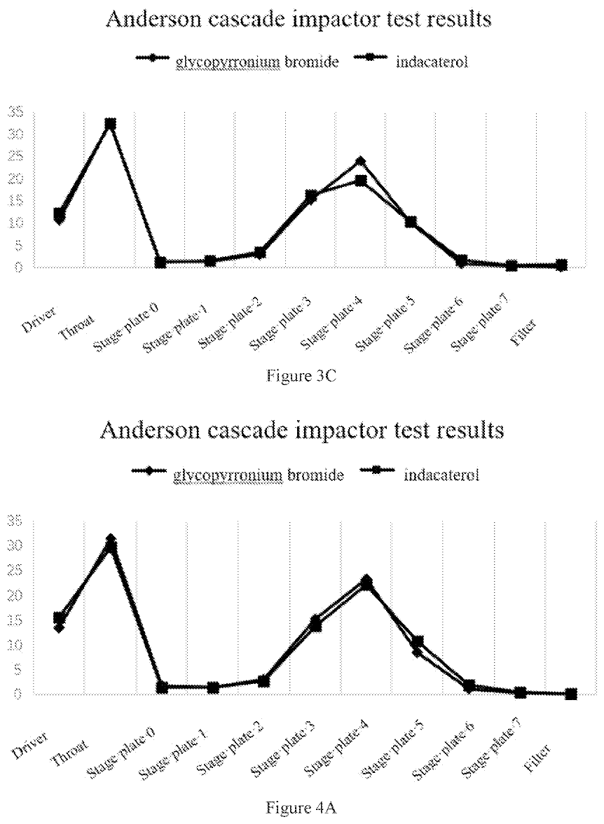

1.5 g glycopyrronium bromide fine powder and 0.3 g indacaterol maleate coarse powder (5:1) were weighed and mixed manually for 10 minutes, then the mixture was added to a jet mill, and micronized at a pressure of 8 bar. The micronized API was sealed and stored until use. 24 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 3A.

0.5 g glycopyrronium bromide fine powder and 0.5 g indacaterol maleate coarse powder (1:1) were weighed and mixed manually for 10 minutes, then the mixture was added to a jet mill, and micronized at a pressure of 8 bar. The micronized API was sealed and stored until use. 24 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 3B.

0.2 g glycopyrronium bromide fine powder and 1.0 g indacaterol maleate coarse powder (1:5) were weighed and mixed manually for 10 minutes, then the mixture was added to a jet mill, and micronized at a pressure of 8 bar. The micronized API was sealed and stored until use. 30 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing. The Anderson cascade impactor test results are shown in FIG. 3C.

TABLE 5
The Anderson cascade impactor test results of glycopyrronium
bromide fine powder and indacaterol maleate coarse powder after
being co-micronized by a jet mill at a pressure of 8 bar

| | FPF of | |
|---|---|---|
| | glycopyrronium | FPF of indacaterol |
| Formulation | bromide (%) | maleate (%) |
| glycopyrronium bromide + | 51.30 | 51.14 |
| indacaterol | | |
| maleate (5:1) | | |
| glycopyrronium bromide + | 55.91 | 56.32 |
| indacaterol | | |
| maleate (1:1) | | |
| glycopyrronium bromide + | 56.90 | 56.13 |
| indacaterol | | |
| maleate (1:5) | | |

As shown in Table 5, as the proportion of glycopyrronium bromide decreases, the dispersibility of glycopyrronium bromide is significantly improved, and the Fine Particle Fraction is increased from 51.30% to 56.90%.

The Anderson test results in FIGS. 3A-3C show that for the aerosols prepared by co-micronizing glycopyrronium bromide fine powder/indacaterol maleate coarse powder at different ratios with a jet mill at a pressure of 8 bar, the degree of co-deposition of glycopyrronium bromide and indacaterol maleate is very high. Among them, the degree of co-deposition of the two active ingredients in each of the aerosols prepared with the mixture of the ratio of 5:1 and 1:1 is higher than that of the ratio of 1:5. This is mainly because the aerosol prepared from glycopyrronium bromide/indacaterol maleate with a ratio of 1:5 has a relatively large difference in the deposition rates of the two active ingredients on the stage plate 4, while the deposition rates on other plates are very similar.

### EXAMPLE 4

0.5 g glycopyrronium bromide coarse powder and 0.5 g indacaterol maleate coarse powder (1:1) were weighed and mixed manually for 10 minutes, and micronized at 10 bar. The API was then sealed and stored until use. 16 mg of the glycopyrronium bromide/indacaterol maleate mixture was weighed, and placed in a 14 mL FCP-coated aluminum can, valve-sealed, filled, ultrasonicated for 10 minutes, and then kept for 2 days for testing.

The pressure of the jet mill was decreased and the above steps were repeated, in order to investigate the effect of pressure (10 bar, 8 bar, 4 bar, 3 bar, 2 bar) on the particle size of the glycopyrronium bromide/indacaterol maleate mixture, the Fine Particle Fraction of glycopyrronium bromide and the degree of co-deposition of the two active ingredients.

TABLE 6
The particle size test results of the glycopyrronium bromide
coarse powder and indacaterol maleate coarse powder mixture

| Airflow | | | | |
|---|---|---|---|---|
| pressure | D10 (μm) | D50 (μm) | D90 (μm) | SPAN |
| 10 bar | 0.61 | 1.42 | 2.86 | 1.58 |
| 8 bar | 0.62 | 1.68 | 3.58 | 1.76 |
| 4 bar | 0.66 | 1.86 | 4.18 | 1.89 |
| 3 bar | 0.67 | 2.31 | 6.35 | 2.46 |
| 2 bar | 0.77 | 3.39 | 9.05 | 2.44 |

The particle size test results in Table 6 show that as the airflow pressure decreases, the particle size of the mixture increases, and the changes in D50, D90 and SPAN are the most significant.

TABLE 7
The Anderson cascade impactor test results of glycopyrronium bromide
coarse powder and indacaterol maleate coarse powder mixture after
being co-micronized by a jet mill at different pressures

| | FPF of | |
|---|---|---|
| | glycopyrronium | FPF of indacaterol maleate |
| Airflow pressure | bromide (%) | (%) |
| 10 bar | 56.31 | 58.31 |
| 8 bar | 54.03 | 56.11 |
| 4 bar | 46.20 | 48.13 |
| 3 bar | 30.04 | 31.32 |
| 2 bar | 14.33 | 12.50 |

The particle size test data and the Anderson cascade impactor test data in Table 6 and Table 7 show that as the airflow pressure increases, the particle size decreases, the dispersibility of glycopyrronium bromide increases, and the FPF increases.

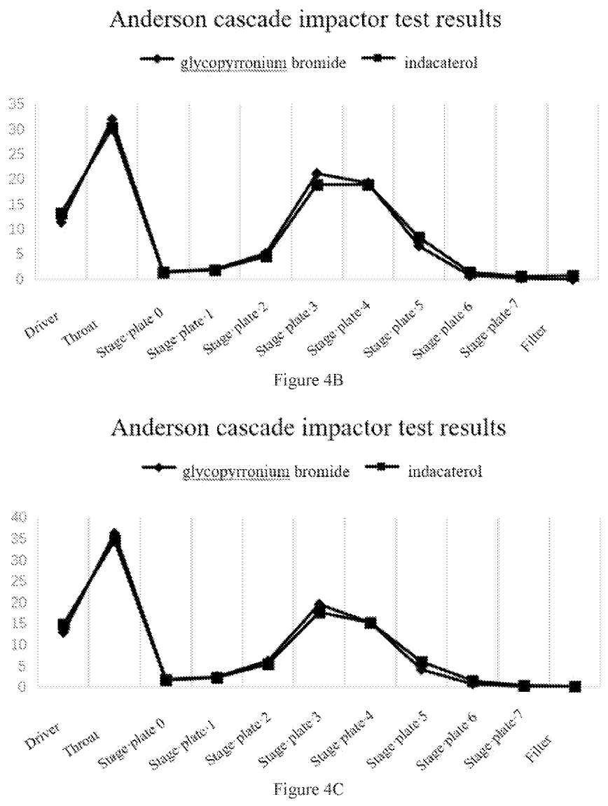

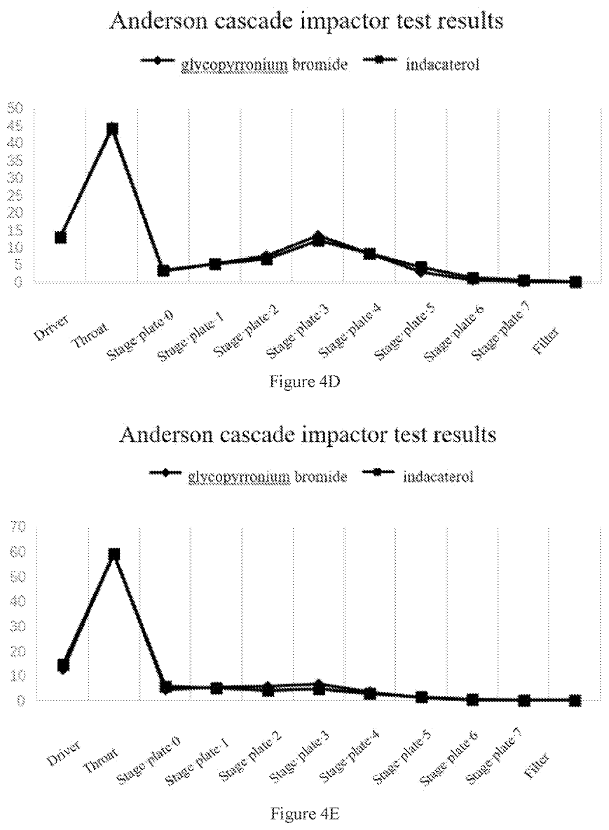

The Anderson cascade impactor test results in FIGS. 4A-4E show that for the aerosols prepared by co-micronizing glycopyrronium bromide coarse powder/indacaterol maleate coarse powder (1:1) with a jet mill at different pressures from 2-10 bar, the degree of co-deposition of glycopyrronium bromide and indacaterol maleate is high. When the airflow pressure is 2 bar or 3 bar, the Fine Particle Fraction of the two active ingredients are very low. Therefore, the pressure of jet-mill micronization is at least 4 bar or more.

### COMPARATIVE EXAMPLE 1

20 mg micronized glycopyrronium bromide was precisely weighed and placed in a 14 mL FCP-coated aluminum can, valve-sealed, filled, ultrasonicated for 10 minutes, and then kept for 2 days for Anderson cascade impactor testing. The specific steps are shown above.

4 mg micronized indacaterol maleate was precisely weighed and placed in a 14 mL FCP-coated aluminum can, valve-sealed, filled, ultrasonicated for 10 minutes, and then kept for 2 days for Anderson cascade impactor testing. The specific steps are shown above.

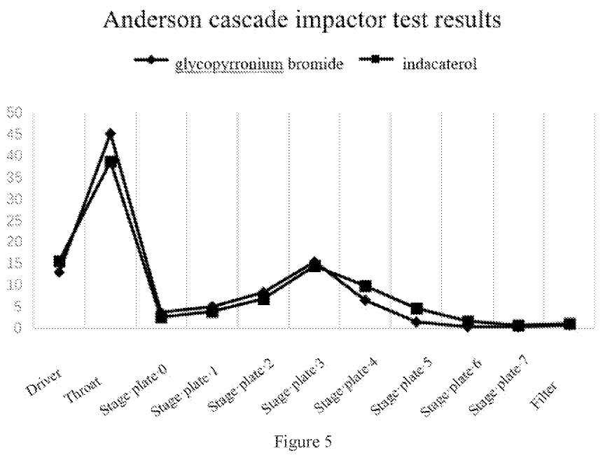

20 mg micronized glycopyrronium bromide and 4 mg micronized indacaterol maleate (5:1) were precisely weighed and mixed manually for 10 minutes. The mixture was then placed in a 14 mL FCP-coated aluminum can, valve-sealed, filled, ultrasonicated for 10 minutes, and then kept for 2 days for Anderson cascade impactor testing. The test results are shown in FIG. 5.

TABLE 8
The Anderson cascade impactor test results of the inhalation
aerosol prepared directly from micronized glycopyrronium
bromide powder and indacaterol maleate powder

| | FPF of glycopyrronium | FPF of indacaterol |
|---|---|---|
| Formulation | bromide (%) | maleate (%) |
| glycopyrronium bromide | 25.68 | — |
| indacaterol maleate | — | 45.66 |
| glycopyrronium | 28.48 | 38.50 |
| bromide:indacaterol (5:1) | | |

As evidenced by the data in Table 8, in the inhalation aerosol prepared directly from micronized glycopyrronium bromide powder and indacaterol maleate powder, the FPF of glycopyrronium bromide has increased from 25.68% of a single formulation to 28.48%, which is only an increase of 3%, and the dispersibility is still very poor. In contrast, the FPF of single indacaterol maleate is 45.66%, and the dispersibility is significantly higher than that of glycopyrronium bromide.

The Anderson test results in FIG. 5 show that the degree of co-deposition of glycopyrronium bromide and indacaterol maleate is very low. In FIG. 5, the ordinate is the deposition rate of the two active ingredients, and the abscissa is the number of each ACI stage plate.

The above are only the preferred embodiments of the present application, so that those skilled in the art can understand or implement the invention of the present application. Various modifications and combinations of these embodiments will be obvious to those skilled in the art, and the general principles defined in this document. can be implemented in other embodiments without departing from the spirit or scope of the present application. Therefore, this application will not be limited to the embodiments shown in this document, but should conform to the widest scope consistent with the principles and novel features disclosed in this document.

The above are only preferred embodiments of the present invention, and do not limit the present invention in any form. Although the present invention has been disclosed as above in preferred embodiments, it is not intended to limit the present invention. Any person familiar with the art, without departing from the scope of the technical solution of the present invention, should be able to use the technical content disclosed above to make some changes or modifications into equivalent embodiments with equivalent changes. Any simple amendments, equivalent changes and modifications made to the above embodiments based on the technical essence of the present invention without departing from the technical solutions of the present invention still fall within the scope of the technical solutions of the present invention.

1. A preparation method of pharmaceutical inhalation aerosol, wherein it comprises the following steps:
(1) mixing glycopyrronium bromide coarse powder with indacaterol fine powder, or glycopyrronium bromide coarse powder with indacaterol coarse powder, or glycopyrronium bromide fine powder with indacaterol coarse powder at a ratio to obtain a glycopyrronium bromide and indacaterol mixture;
(2) micronizing, by a crushing device under pressure, the glycopyrronium bromide and indacaterol mixture prepared in step (1) to obtain a micronized glycopyrronium bromide and indacaterol mixture; and
(3) adding the micronized glycopyrronium bromide and indacaterol mixture prepared in step (2) to an aluminum can, performing valve sealing, and filling with a propellant.

2. The method of claim 1, wherein in the step (1), the ratio is 5:1 to 1:5 by mass.

3. The method of claim 2, wherein the ratio is 1:1 to 1:5 by mass.

4. The method of claim 1, wherein in the step (2), the pressure of the micronization is 4-10 bar.

5. The method of claim 4, wherein in the step (2), the pressure of the micronization is 8-10 bar.

6. The method of claim 1, wherein in the step (2), the feed rate of the glycopyrronium bromide and indacaterol mixture is 0.5-1.0 g/min.

7. The method of claim 6, wherein in the step (2), the feed rate of the glycopyrronium bromide and indacaterol mixture is 0.5 g/min.

8. The method of claim 1, wherein in the step (2), the crushing device is a ball mill, a jet mill, a high-pressure homogenizer, or a spray dryer.

9. The method of claim 1, wherein in the step (3), the propellant is one of HFA 134a, HFA 227, HFA 152.

10. The method of claim 1, wherein in the step (3), the propellant is a mixture of HFA 134a, HFA 227, and HFA 152.

11. The method of claim 1, wherein in the step (2), the particle size D90 distribution range of the micronized glycopyrronium bromide and indacaterol mixture is 2.86 μm to 4.18 μm, the D50 distribution range is 1.42 μm to 1.86 μm, and the D10 distribution range is 0.58 μm to 0.66 μm.

12. The method of claim 11, wherein the particle size D90 distribution range of the micronized glycopyrronium bromide and indacaterol mixture is 2.86 μm to 3.58 μm, the D50 distribution range is 1.42 μm to 1.68 μm, and the D10 distribution range is 0.58 μm to 0.62 μm.

13. The method of claim 12, wherein the particle size D90 distribution range of the micronized glycopyrronium bromide and indacaterol mixture is 2.86 μm to 3.40 μm, the D50 distribution range is 1.42 μm to 1.53 μm, and the D10 distribution range is 0.58 μm to 0.61 μm.

14. The method of claim 1, wherein in the step (1), the ratio is 1:5 by mass; and in the step (2), the pressure of the micronization is 10 bar.

15. A pharmaceutical inhalation aerosol prepared according to the method of claim 1, wherein it comprises glycopyrronium bromide, indacaterol, and a propellant.

16. The pharmaceutical inhalation aerosol of claim 15, wherein the FPF range of the glycopyrronium bromide is 35% to 60%, and the FPF range of the indacaterol is 35% to 65%.

17. The pharmaceutical inhalation aerosol of claim 16, wherein the FPF range of the glycopyrronium bromide is 45% to 60%, and the FPF range of the indacaterol is 40% to 60%.

18. The pharmaceutical inhalation aerosol of claim 17, wherein the FPF range of the glycopyrronium bromide is 55% to 60%, and the FPF range of the indacaterol is 55% to 60%.
